(19) United States
(12) Patent Application Publication
(10) Pub. No.: US 2010/0099127 A1
(43) Pub. Date: Apr. 22, 2010

(54) SYSTEM AND METHOD FOR ACQUIRING AND STORING BIOLOGICAL SAMPLES

(75) Inventor: Moishe Bodner, Brooklyn, NY (US)

Correspondence Address: Kaplan Gilman & Pergament LLP, 1480 Route 9 North, Woodbridge, NJ, 07095, US

(73) Assignee: StatSure Diagnostic Systems, Inc., Brooklyn, NY (US)

(21) Appl. No.: 12/255,173

(22) Filed: Oct. 21, 2008

(51) Int. Cl. C12Q 1/40; C12M 1/28

(52) U.S. Cl. 435/22; 435/309.1

(57) ABSTRACT

A system and method that may include grasping a sample collection medium with a holder; inserting the sample collection medium into a medium storage device within a first container such that at least a first portion of the sample collection medium is securely held within the first container; separating a second portion of the sample collection medium retained within the holder from the first portion of the sample collection medium held within the first container; and inserting the second portion of the sample collection medium into a second container.

### BACKGROUND OF THE INVENTION

This invention relates in general to obtaining biological samples for testing purposes and in particular to enabling verification of biological sample test results.

Various devices have been employed to collect and store biological samples from people for various purposes such as testing for diseases and for drugs. In some situations, test results obtained from a stored biological sample may be challenged based the contention that incriminating material arose from improper testing procedure, rather than from the test subject.

Currently, when the results of a drug test are challenged, a testing service can obtain another biological sample (such as, saliva, blood, or other substance) from the test subject, transport the newly obtained sample to a testing lab, and repeat the analysis of the sample. If the second analysis confirms the results of the first analysis, the matter may be considered resolved.

However, the process of repeating the steps of obtaining a further biological sample, transporting the sample to a lab, and examining the additional sample is time consuming and expensive. Accordingly, an improved method for verifying the results of drug tests is needed.

### SUMMARY OF THE INVENTION

According to one aspect, the invention is directed to an apparatus for holding a biological sample comprising a sample collection medium; a medium holder for holding the sample collection medium; and a container for housing the holder, the container including a medium storage device for collecting the sample collection medium, wherein the medium storage device is operable to retain a first portion of the sample collection medium, upon removal of second portion of the sample collection medium through manipulation of the holder.

Other aspects, features, advantages, etc. will become apparent to one skilled in the art when the description of the preferred embodiments of the invention herein is taken in conjunction with the accompanying drawings.

### BRIEF DESCRIPTION OF THE DRAWINGS

For the purposes of illustrating the various aspects of the invention, there are shown in the drawings forms that are presently preferred, it being understood, however, that the invention is not limited to the precise arrangements and instrumentalities shown.

FIG. 1 is an elevational view of a device for gathering a biological sample in accordance with an embodiment of the present invention;

FIG. 2 is an elevational view of the device of FIG. 1 in which a sample collection medium has been separated into two parts, in accordance with an embodiment of the present invention;

FIG. 3 is an elevational view of a holder inserting a portion of a sample collection medium into a second container, in accordance with an embodiment of the present invention;

FIG. 4 depicts an alternative embodiment in which a spring like member is used to secure the sample collection medium; and

FIG. 5 shows an additional embodiment of the present invention.

### DETAILED DESCRIPTION OF THE PREFERRED EMBODIMENTS

In the following description, for purposes of explanation, specific numbers, materials and configurations are set forth in order to provide a thorough understanding of the invention. It will be apparent, however, to one having ordinary skill in the art that the invention may be practiced without these specific details. In some instances, well-known features may be omitted or simplified so as not to obscure the present invention. Furthermore, reference in the specification to phrases such as “one embodiment” or “an embodiment” means that a particular feature, structure or characteristic described in connection with the embodiment is included in at least one embodiment of the invention. The appearances of phrases such as “in one embodiment” or “in an embodiment” in various places in the specification do not necessarily all refer to the same embodiment.

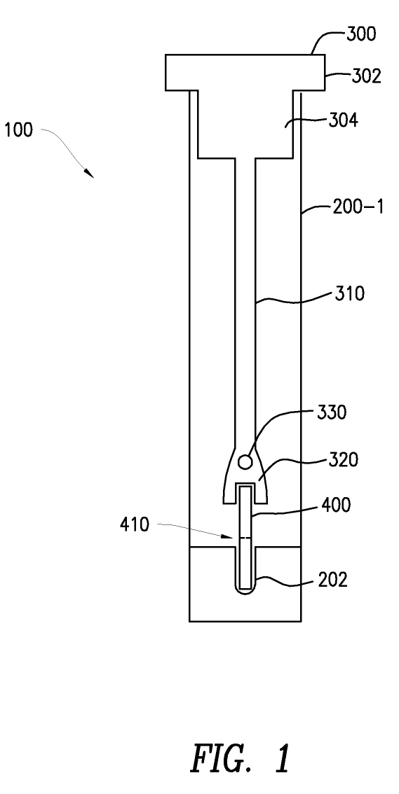

FIG. 1 is an elevational view of a device for gathering a biological sample in accordance with an embodiment of the present invention. Biological sampling device 100 may include container 200 and holder 300. Container 200 may include medium storage device 202. Holder 300 may include shaft 310, gripping mechanism 320, and/or sample adequacy indicator 330. Sample collection medium 400 may include portions 400-a and 400-b and/or disassembly mechanism 410. Sample adequacy indicator 330 may employ the saliva adequacy system disclosed in U.S. Pat. No. 5,268,148 which is hereby incorporated by reference herein in its entirety.

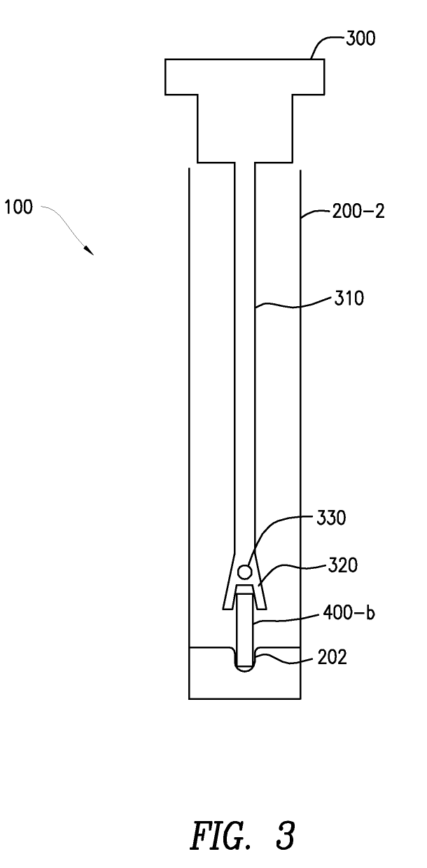

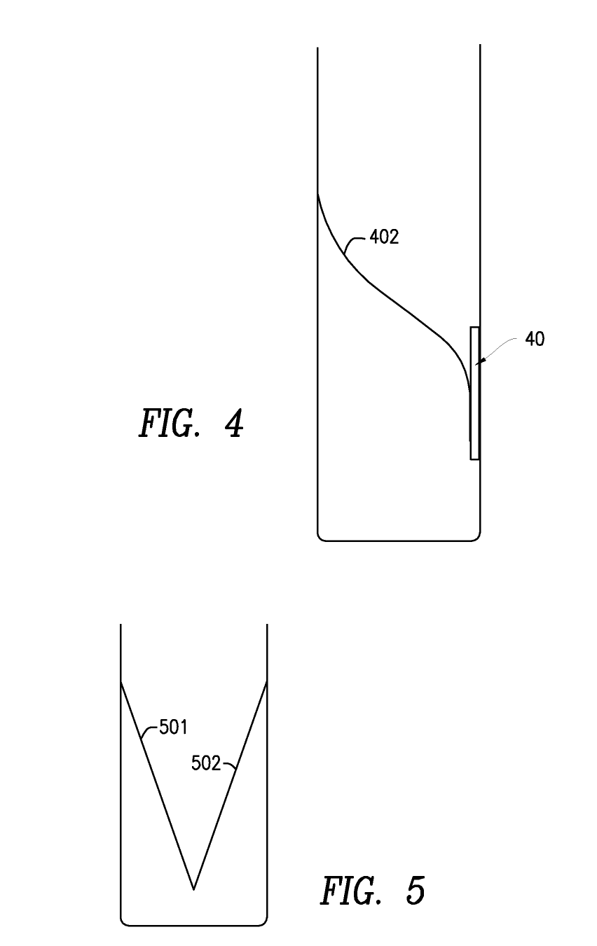

Container 200 may be made of plastic and may be a substantially hollow tube of circular cross section. Container 200 may serve as a container for biological samples, such as saliva, blood, tissue, or other biological material, and is therefore preferably configured to be suitably insulated against contamination from materials outside container 200. Container 200 may include medium storage device 202 which may include a substantially planar sheet of plastic having a U-shaped cutout (or other suitable cutout shape) therein, as shown in FIGS. 1-3. The U-shaped cutout is preferably operable to store sample collection medium 400 when placed therein, and/or to retain a portion 400-a of medium 400 when another portion 400-b of medium 400 is removed from container 100, as discussed in greater detail below.

Medium holder 300 is preferably made of plastic, though other materials may be employed. Holder 300 may include a cap 302, and an interface portion 304, which may be threaded. Holder 300 may further include gripping mechanism 320 which is preferably configured for securely holding a sample collection medium 400. Holder 300 may further include a sample adequacy indicator 330 that is preferably operable to indicate when collection medium 400 has received a sufficient quantity of testing material, such as, for instance, saliva, blood, or other testing material for testing to be properly performed on the material.

Sample collection medium 400 may include disassembly mechanism 410 which may facilitate the separation of medium 400 into portions 400-a and 400-b, and/or still further portions. Disassembly mechanism may be a perforation. However, mechanisms other than perforations may be employed for this purpose. Medium 400 may be made of cotton, cloth, and/or other materials suitable for receiving and storing fluid or tissue samples for storage in container 200 and subsequent testing.

A preferred embodiment of present invention is preferably operable to a single biological sample, such as saliva, taken from a test subject and received onto a sample collection medium 400 to be divided into two or more sample collection media portions and stored in separate containers. The above process preferably enables multiple redundant testing operations on an original sample to be conducted without having to acquire a new biological sample from the test subject.

In a preferred embodiment, a sample of biological material, such as saliva, may be obtained and absorbed into, or stored on, sample collection medium 400. Holder 300 may be used to grasp collection medium 400 and place the same within container 200-1. More specifically, holder 300 may be used to insert sample collection medium into a medium storage device 202 within container 200.

FIG. 1 shows sample collection medium 400 fully inserted into medium storage device 202 within container 200. The portion of collection medium 400 located within the U-shaped cutout in medium storage device 202 preferably experiences a snug fit. Holder 300 may then be twisted about its longitudinal axis while being withdrawn from container 200 to thereby separate portion 400-a and portion 400-b of sample collection medium 400.

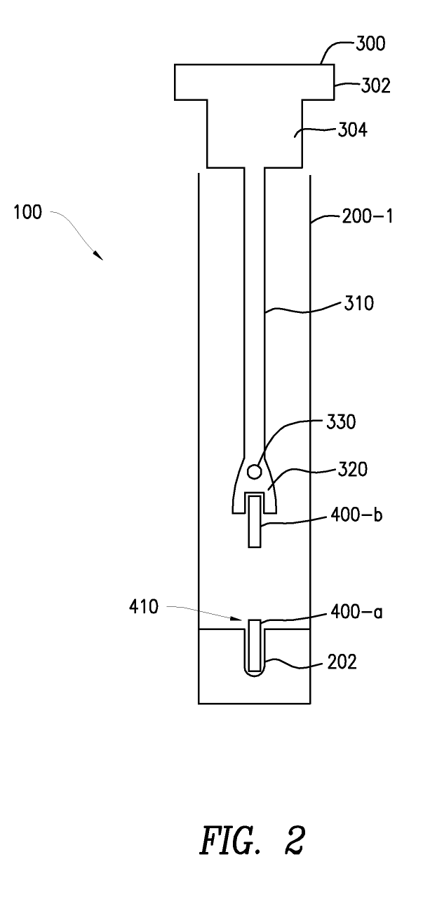

FIG. 2 shows portion 400-a lodged within medium storage device 202, with 400-b still lodged within gripping mechanism 320 of holder 300.

After having separated portion 400-a and 400-b, holder 300 may be completely removed from container 200-1 and inserted into container 200-2. Portion 400-b of sample collection medium 400 may then be lodged within medium storage device 202 of container 200-2, and holder 300 may thereafter be removed from container 200-2.

Thus, at the conclusion of the insertion operations for the two portions of sample collection medium 400, two separate portions 400-a and 400-b of a single original sample collection medium 400 are securely stored in two separate respective containers 200-1 and 200-2. This approach makes redundant testing material available without having to the repeat the steps of acquiring biological material from a test subject, and transporting the biological material to a test location.

In the foregoing, the medium storage device 202 is disclosed as including a U-shaped cutout for securing one portion 400-a of the sample collection medium 400, as the holder 300 and another portion 400-b of the medium are removed from container 200-1. However, the present invention is not limited to using the U-shaped cutout as a means for securing portion 400-a of collection medium 400. In other embodiments, alternative structures could be employed for this purpose, including but not limited to cutouts having a square or rectangular shape.

Moreover, devices employing spring loading could be used to help secure a first portion 400-a of the sample collection medium 400 as a second portion 400-b of the sample collection medium is removed. In one embodiment, spring-biased elements could be included within medium storage device 202, and sample collection medium 400 could be partially inserted between the spring-biased parts when initially inserting medium 400 into container 200-1. In other embodiments, a grasping mechanism could be employed, in which an operator moves a switch, knob, or other control device to securely lodge a first portion 400-a of sample collection medium 400 within medium storage device 202.

Still another embodiment depicts a spring like member 402 which leaves a tiny space 401 into which the sample collection medium is inserted. When the medium is pulled, member 402 causes the sample collection medium to get stuck, and tear at the weakest point. By perforating or otherwise weakening the sample collection medium at the proper place, the medium can be divided cut where needed.

FIG. 5 depicts still another embodiment of the invention, wherein two spring-like members 501 and 502 protruding from opposite sides of the tube and facing downwardly are used. The collection medium can be easily inserted in the space between 501 and 502 due to the flimsy, resilient nature of these members, but it locks and can not be pulled upwardly, tearing at the predetermined weakened part.

In another embodiment, the present invention can be used with a sample collection device that has an adequacy indicator, such as that described in U.S. Pat. No. 5,268,148. (“The '148 patent”). The '148 patent is hereby incorporated by reference in its entirety.

In the foregoing, disassembly mechanism 410 of sample collection medium 400 is disclosed as optionally including a perforation for facilitating the separation of portion 400-a from portion 400-b of sample collection medium 400. However, the present invention is not limited to this embodiment. Other disassembly mechanisms may be employed in place of, or in addition to, a perforation.

Although the invention herein has been described with reference to particular embodiments, it is to be understood that these embodiments are merely illustrative of the principles and applications of the present invention. It is therefore to be understood that numerous modifications may be made to the illustrative embodiments and that other arrangements may be devised without departing from the spirit and scope of the present invention as defined by the appended claims.

1. Apparatus for holding a biological sample
a sample collection medium;
a medium holder for holding the sample collection medium; and
a container for housing the holder, the container including a medium storage device for collecting the sample collection medium,
wherein the medium storage device is operable to retain a first portion of the sample collection medium, upon removal of second portion of the sample collection medium through manipulation of the holder.

2. The apparatus of claim 1 wherein the sample collection medium includes a disassembly mechanism.

3. The apparatus of claim 2 wherein the disassembly mechanism is a perforation within the sample collection medium.

4. The apparatus of claim 1 wherein the medium holder is configured to continue to grasp the sample collection medium even during separation of the first portion of the medium form the second portion of the medium.

5. The apparatus of claim 1 wherein the medium storage device includes a cavity suitable for receiving and retaining the first portion of the sample collection medium.

6. The apparatus of claim 1 wherein the medium storage device in the first container includes a springingly biased member for securing the first portion of the sample collection medium against a rigid portion of the container.

7. The apparatus of claim 1 wherein the medium storage device in the first container includes a grasping mechanism for securing the first portion of the sample collection medium against a rigid portion of the container.

8. The apparatus of claim 7 wherein the grasping mechanism is controllable by a human operator.

9. The apparatus of claim 1 wherein the medium holder comprises a sample adequacy indicator operable to indicate when a sufficient quantity of a material for testing has been collected by the sample collection medium.

10. The apparatus of claim 9 wherein the sample adequacy indicator indicates sufficiency of the collected material by changing from a first color to a second color.

11. A method, comprising
grasping a sample collection medium with a holder;
inserting the sample collection medium into a medium storage device within a first container such that at least a first portion of the sample collection medium is securely held within the first container;
separating a second portion of the sample collection medium retained within the holder from the first portion of the sample collection medium held within the first container; and
inserting the second portion of the sample collection medium into a second container.

12. The method of claim 11 further comprising:
collecting a biological sample from a test subject onto the sample collection medium; and
discontinuing the collecting step once a sample adequacy indicator indicates that a sufficient quantity of the biological sample has been collected.

13. The method of claim 12 wherein the biological sample is saliva and the sample adequacy indicator is a saliva adequacy indicator.

14. The method of claim 12 wherein the sample adequacy indicator indicates sufficiency of the collected quantity by changing from a first color to a second color.

15. The method of claim 11 further comprising:
securely holding the first portion of the sample collection medium within the first container using a shaped opening within a sheet of material within the first container.

16. The method of claim 11 further comprising:
securely holding the first portion of the sample collection medium within the first container using a member that is springingly biased so as to bind the first portion of the sample collection medium against a rigid portion of the container.

17. The method of claim 11 further comprising:
securely holding the first portion of the sample collection medium within the first container using a grasping mechanism.

18. The method of claim 17 wherein said grasping mechanism includes at least one spring like member facing downwardly.

19. The method of claim 17 wherein said grasping mechanism contains at least two resilient members.
